(19) United States
(12) Patent Application Publication
(10) Pub. No.: US 2020/0289379 A1
(43) Pub. Date: Sep. 17, 2020

(54) ORAL CARE COMPOSITIONS AND METHODS OF USE

(71) Applicant: Bernardino Reynoso, Brooklyn, NY (US)

(72) Inventor: Bernardino Reynoso, Brooklyn, NY (US)

(21) Appl. No.: 16/758,006

(22) PCT Filed: Oct. 25, 2018
(86) PCT No.: PCT/US2018/057489

§ 371 Date: Apr. 21, 2020

Related U.S. Application Data
(60) Provisional application No. 62/577,629, filed on Oct. 26, 2017.

(51) Int. Cl. A61K 8/02; A61K 8/34; A61K 8/9789; A61K 8/81; A61K 8/90; A61Q 11/00

(52) U.S. Cl. CPC A61K 8/0233; A61K 8/345; A61K 8/9789; A61K 8/8147; A61K 8/8152; A61K 2800/222; A61Q 11/00; A61K 2800/31; A61K 2800/5424; A61K 2800/28; A61K 2800/62; A61K 8/90

(57) ABSTRACT

The described invention relates, in part, to a water-free, toothbrush-free oral composition in the form of a tablet, comprising neem, xylitol, one or more polymers, optionally magnolia bark extract, and a coating, wherein the tablet is activated in situ by a chewing action and/or by contacting saliva, and methods of use.

### RELATED APPLICATION

The present application claims priority to U.S. Provisional Application No. 62/577,629, filed on Oct. 26, 2017, the entire content of which is incorporated by reference in its entirety herein.

### BACKGROUND

Oral care is an indicator of a body's overall health, but for many of the world's poor, oral health care is one of the most neglected areas of medical care available (World Health Organization). Worldwide 60-90% of school children and nearly 100% of adults have dental cavities. There is a direct correlation between high poverty and poor dental health. While toothpaste and toothbrushes are the most common oral hygiene products, in developing countries where water supply is erratic, not clean, or toothbrushes and toothpaste are expensive, there is a need to develop an oral hygiene product that is usable where there may not be water, toothpaste or other necessary elements for oral care.

A tooth is comprised of an inner dentin layer and an outer hard enamel layer that is the protective layer of the tooth. The enamel layer of a tooth is naturally opaque, and white or a slightly off-white color. The enamel layer is composed of hydroxyapatite mineral crystals that create a somewhat porous surface. These hydroxyapatite crystals form microscopic hexagonal rods or prisms that make up the enamel surface. As a result, the surface of the enamel presents microscopic spaces or pores between the prisms. Without limiting the mechanism, function or utility of the described invention, it is believed that the porous nature of the enamel at least in part enables discoloring substances to permeate the enamel and discolor the teeth.

Dental plaque is present to some degree in the form of a film on dental surfaces. It is a byproduct of microbial growth, and comprises a dense microbial layer consisting of a mass of microorganisms embedded in a polysaccharide matrix. It is reported that plaque adheres firmly to dental surfaces and is removed only with difficulty even through a rigorous brushing regimen. Moreover, plaque rapidly re-forms on the tooth surface after it is removed. Plaque may form on any part of the tooth surface, and is found particularly at the gingival margin, in cracks in the enamel, and on the surface of dental calculus. The problem associated with the formation of plaque on the teeth lies in the tendency of plaque to build up and eventually produce gingivitis, periodontitis and other types of periodontal disease, as well as dental caries, bad breath (halitosis) and dental calculus.

As plaque is formed by oral bacteria, a wide variety of antibacterial agents have been proposed to retard plaque formation and the oral infections associated with plaque formation. For example, halogenated hydroxydiphenyl ether compounds are well known in the art for their antibacterial activity, and have been used in oral compositions to counter plaque formation by bacterial accumulation in the oral cavity.

### SUMMARY OF THE DISCLOSURE

The described invention relates to oral care compositions. According to one aspect, the described invention provides an oral care composition in the form of a tablet, comprising neem; xylitol; and one or more carboxylic block copolymers, wherein the tablet is activated in situ by a chewing action and/or by contacting saliva. According to another aspect, the described invention provides an oral care composition in the form of a tablet, comprising coconut oil; xylitol; and one or more polymers or copolymers, wherein the tablet is activated in situ by a chewing action and/or by contacting saliva. According to one embodiment, the tablet is chewable. According to another embodiment, the oral care composition further comprises an effervescent agent. According to another embodiment, the oral care composition is digestible. According to one embodiment, the polymer is a block copolymer. According to another embodiment, the block copolymer is a carboxylic acid copolymer. According to another embodiment, the carboxylic block polymer is Gantrez®S-97BF. According to another embodiment, the polymer comprises an anionic polymer or anionic copolymer. According to another embodiment, the anionic polymer comprises acrylic acid or an ester of acrylic acid. According to another embodiment, the anionic polymer comprises methacrylic acid or an ester of methacrylic acid. According to another embodiment, the anionic polymer is a copolymer of acrylic acid and methacrylic acid. According to another embodiment, the anionic polymer is a copolymer of an ester of acrylic acid and an ester of methacrylic acid. According to another embodiment, the oral care composition further comprises an abrasive. In a further embodiment, the abrasive is a mineral abrasive. In a further related embodiment, the abrasive is selected from the group consisting of: silicic acids, calcium carbonates, calcium phosphates, aluminum oxides and/or hydroxyapatites, sodium metaphosphate, potassium metaphosphate, tricalcium phosphate, dihydrated dicalcium phosphate, micronized silicon, aluminum silicate, calcined alumina, bentonite, surface-active substances (e.g., sodium lauryl sulfate, sodium lauryl sarcosinate, and cocamidopropylbetaine), and other siliceous materials, and combinations thereof. According to another embodiment, the oral care composition further comprises charcoal. According to one embodiment, the oral care composition further comprises an antibacterial plant extract. According to a further embodiment, the antibacterial plant extract is magnolia bark or magnolia bark extract. According to one embodiment, the tablet comprises a contoured surface. According to some embodiments, the contoured surface is effective to cause the tablet's edges to move around the oral cavity. According to one embodiment, the tablet is sized from about 0.25 inches to about 0.75 inches in diameter. According to another embodiment, thickness of the tablet is sized from about 0.1 inches to about 0.5 inches thick. According to another embodiment, the tablet is sized at about 0.5 inches in diameter and 0.25″ thick. According to another embodiment, the tablet comprises a coating. According to one embodiment, the coating comprises an anionic polymer or copolymer. According to one embodiment, the coating comprises about 2%, 2.1%, 2.2%, 2.3%, 2.4%, 2.5%, 2.6%, 2.7%, 2.8%, 2.9%, 3.0%, 3.1%, 3.2%, 3.3%, 3.4%, 32.5%, 3.6%, 3.7%, 3.8%, 3.9%, 4%, 4.1%, 4.2%, 4.3%, 4.4%, 4.5%, 4.6%, 4.7%, 4.8%, 4.9% or 5% anionic polymer or copolymer by total weight. According to another embodiment, the anionic polymer comprises acrylic acid, or methacrylic acid. According to another embodiment, the anionic polymer is a copolymer of acrylic acid and methacrylic acid, a copolymer of esters of acrylic acid, a copolymer of esters of methacrylic acid, or a copolymer of esters of acrylic acid and esters of methacrylic acid. According to one embodiment, the neem, xylitol and one or more polymers are distributed evenly throughout the tablet. According to one embodiment, the neem, xylitol and one or more polymers are distributed in layers throughout the tablet. According to another embodiment, the oral care composition further comprises one or more of a flavorant, a pigment, a dye, a whitening agent, an anti-tartar agent, a desensitizing agent, a sensate, a vitamin, a preservative, an enzyme, saliva-stimulating agent, or a mixture thereof.

According to another aspect, the described invention features a method for cleaning surfaces of the oral cavity comprising contacting the oral cavity with the oral care composition of any of the aspects and embodiments described herein. According to one embodiment, the oral cavity includes the teeth, the tongue, the gums and the cheeks.

According to another aspect, the described invention provides a method for the treatment or prevention of enamel erosion on a dental surface, comprising contacting the dental surface with the oral care composition of any of the aspects and embodiments described herein.

According to another aspect, the described invention provides a method for the treatment or inhibition of a chemical stain, plaque, and/or tartar on a dental surface, comprising contacting the dental surface with the oral care composition of any of the aspects and embodiments described herein, wherein the method provides a sensorial feel of a clean mouth.

According to another aspect, the described invention provides a method for whitening the teeth, comprising contacting the dental surface with the oral care composition of any of the aspects and embodiments described herein.

According to another aspect, the described invention provides a method for the treatment or inhibition of gum disease comprising contacting the oral cavity with the oral care composition of any of the aspects and embodiments described herein.

According to another aspect, the described invention provides a method for the treatment or inhibition of halitosis comprising contacting the oral cavity with the oral care composition of any of the aspects and embodiments described herein.

According to another aspect, the described invention provides a method for the inhibition of biofilm formation on a dental surface comprising contacting the oral cavity with the oral care composition of any of the aspects and embodiments described herein.

According to another aspect, the described invention provides a method for the treatment or inhibition of bacteria from sticking together and growing into bigger colonies in an oral cavity comprising contacting the oral cavity with the oral care composition of any of the aspects and embodiments described herein.

### BRIEF DESCRIPTION OF THE DRAWINGS

FIG. 1 is a schematic that shows the top of front views of the oral composition in the form of a tablet according to exemplary embodiments.

FIG. 2 is a schematic that shows various layers of the oral composition in the form of a tablet according to exemplary embodiments.

### DETAILED DESCRIPTION OF THE DISCLOSURE

The described invention provides, in part, an oral composition in the form of a tablet, comprising neem, xylitol, and one or more polymers, wherein the tablet is activated in situ by a chewing action and/or by contacting saliva.

Without being bound by theory, the ingredients of the tablets of the described invention effect their oral hygiene properties by bonding to oral surfaces while activating the saliva to maintain fresh breath for longer hours. The tablets of the disclosure address the need for better oral care hygiene where there is lack of funds, clean water, lack of oral care health professionals or care in general, or education on oral hygiene. The tablets of the disclosure can also be used, for example, in schools and other facilities to allow for maintenance and upkeep of healthier lifestyle and better oral hygiene.

Further areas of applicability of the described invention will become apparent from the detailed description provided herein. It should be understood that the detailed description and specific examples, while indicating the preferred embodiment of the disclosure, are intended for purposes of illustration only and are not intended to limit the scope of the disclosure.

### I. Definitions

As used throughout, ranges are used as shorthand for describing each and every value that is within the range. Any value within the range can be selected as the terminus of the range. In addition, each reference cited herein is hereby incorporated by referenced in its entirety. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

As used herein, the term “antibacterial” is meant to refer to inhibition or arrest of the growth of a bacterium, or a reduction in the severity of or likelihood of developing a bacterial disease, inducing death of the bacterium, or reduction or inhibition of the pathogenic effects of the bacterium.

As used herein, the term “biofilm” refers to a slimy, gluelike substance formed from the attachment of microorganisms to surfaces and the subsequent development of multiple layers of cells. Biofilms can be formed by a single bacterial species, but more often are formed from many species of microorganisms. Essentially, a biofilm may form on any surface exposed to bacteria and some amount of water.

As used herein, the term “block copolymer” refers to a polymer containing long stretches of two or more monomeric units linked together by chemical valences in a single chain.

As used herein, the term “block polymer” refers to a polymer whose molecule is made up of alternating sections of one chemical composition separated by sections of a different chemical nature or by a coupling group of low molecular weight.

As used herein, the term “carboxylic acid” refers to any of a broad array of organic acids comprised of alkyl groups, for example, in a straight chain (aliphatic), terminating in a carboxyl group (COOH). Carbon atoms near the carboxyl group are often designated by Greek letters. The atom adjacent to the carbonyl function is alpha, the next removed is beta, and so on. The number of carbon atoms can range from one to 26. Carboxylic acids may be saturated or unsaturated.

As used herein, “chemical stain” refers to a discoloration of a dental surface caused by adsorption or absorption of a colored agent on or into the surface, or caused by chemical reaction of material of the dental surface (e.g., dental enamel) with a colored or noncolored agent contacting the surface. “Chemical staining” is used to mean formation and/or development of a chemical stain.

The term “condition”, as used herein, refers to a variety of health states and is meant to include disorders or diseases caused by any underlying mechanism or disorder, or injury, and the promotion of healthy tissues and organs.

The term “contact” and its various grammatical forms as used herein refers to a state or condition of touching or of immediate or local proximity.

As used herein, the term “copolymer” is meant to refer to a polymer produced by the simultaneous polymerization of two or more dissimilar monomers.

As used herein, the term “dental plaque” refers to the diverse microbial community (predominantly bacteria) found on the tooth surface, embedded in a matrix of polymers of bacterial and salivary origin. Plaque is a form of biofilm.

As used herein, “dental surface” refers to a surface of a natural tooth or a hard surface of artificial dentition including a denture, dental plate, crown, cap, filling, bridge, dental implant and the like. According to some embodiments, the dental surface is a natural tooth.

The term “disease” or “disorder”, as used herein, refers to an impairment of health.

As used herein, “effervescent” or “effervescence” refers to emitting small bubbles of gas continuously produced for a period of time.

As used herein, the term “extract” and its various grammatical forms is used to mean a mean a product (an extract) resulting from a chemical or physical process (extraction) that dissolves, withdraws, distills or otherwise separates out certain constituents of a parent mixture from its other constituents.

As used herein, the term “inhibit” refers to a decrease compared to a control or a decrease compared to a subject prior to administration of the oral care compositions of the described invention.

As used herein, the term “molecular weight” is meant to refer to a weight average molecular weight (MW) unless otherwise indicated.

As used herein, an “oral care composition” refers to a composition for which the intended use can include oral care, oral hygiene, or oral appearance, or for which the intended method of use can comprise administration to the oral cavity. According to some embodiments, an oral care composition is retained in the oral cavity for a time sufficient to effect the intended utility. The oral care compositions as disclosed herein may be used in nonhuman mammals such as companion animals (e.g., dogs and cats), as well as by humans as long as the formulation is adjusted to be nontoxic to those animals. According to some embodiments, the oral care compositions as disclosed herein are used by humans.

As used herein, the term “polymer” refers to any of various chemical compounds made of smaller, identical molecules (called monomers) linked together. Polymers generally have high molecular weights. The process by which molecules are linked together to form polymers is called “polymerization.”

As used herein, the term “treat” or “treating” includes preventing, alleviating, ameliorating, inhibiting, or mitigating. The term “treat” or “treating” as used herein refers to accomplishing one or more of the following: (a) reducing the severity of a disorder; (b) limiting the development of symptoms characteristic of a disorder being treated; (c) limiting the worsening of symptoms characteristic of a disorder being treated; (d) limiting the recurrence of a disorder in patients that previously had the disorder; and (e) limiting recurrence of symptoms in patients that were previously asymptomatic for the disorder.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

All percentages, parts and ratios as used herein are by weight of the total dosage form, unless otherwise specified. All such weights as they pertain to listed ingredients are based on the active level and, therefore do not include solvents or by-products that may be included in commercially available materials, unless otherwise specified.

### II. Oral Care Compositions

According to one aspect, the described invention provides oral care compositions in the form of a tablet comprising neem; xylitol; and one or more polymers, wherein the tablet is activated in situ by a chewing action and/or by contacting saliva. According to another aspect, the described invention provides oral care compositions in the form of a tablet, comprising coconut oil; xylitol; and one or more polymers, wherein the tablet is activated in situ by a chewing action and/or by contacting saliva. According to one embodiment, the polymer is a block copolymer. According to some embodiments, the block copolymer comprises at least one hydrophobic block and at least one hydrophilic block.

According to another embodiment, the block copolymer is a carboxylic acid block copolymer. According to another embodiment, the carboxylic acid block polymer is Gantrez®S-97BF.

According to some embodiments, the polymer comprises an anionic polymer or copolymer. According to another embodiment, the anionic polymer or copolymer comprises acrylic acid:

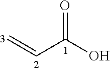

or an ester of acrylic acid: According to some embodiments, the anionic polymer comprises methacrylic acid:

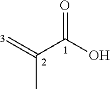

or an ester of methacrylic acid. According to some embodiments, the anionic polymer is a copolymer of acrylic acid and methacrylic acid or a copolymer of an ester of acrylic acid and an ester of methacrylic acid. Exemplary esters of general formula RCOOR′ include, without limitation, those in which R′ is CO—O—CH₂—CH₂N(CH₃)₂; CO—OCH₃; COOH; or CO—OCH₃CH₂N(CH₃)₂⁺Cl⁻.

According to some embodiments, the anionic polymer is a copolymer derived from esters of acrylic and methacrylic acid, which is commercially available from Evonik Industries as EUDRAGIT®.

According to one embodiment, the tablet is a chewable tablet. According to one embodiment, the tablet is digestible. According to one embodiment, the tablet is not taken with water or dissolved in water. According to one embodiment, the tablet is packaged in a recyclable package. According to one embodiment, the recyclable package is a recyclable single-use sachet.

### Neem

The plant Azadirachta indica is more commonly known as the neem tree. It has been used as a major component in traditional Indian medicine (Ayurvedic medicine). Its applications have been varied; for example, it has been used to treat skin disease and as a biopesticide. Its twigs have an ability to inhibit S. mutans. Without being bound by theory, azadirachtin and nimbin, chemical compounds found in neem, can have antimicrobial properties.

According to one embodiment, neem is present in the oral care composition in an amount of from about 0.5% to about 95%, by total weight of the oral care composition, for example 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, by total weight of the oral care composition.

### Magnolia Bark Extract

According to some embodiments, the oral care composition comprises an antibacterial plant extract. According to some embodiments, the antibacterial plant extract has antimicrobial activity for oral or periodontal pathogens. According to some embodiments, the antibacterial agent is magnolia bark extract (MBE). According to some embodiments, the MBE has anti-inflammatory activity. According to some embodiments, an extract of magnolia is an extract from dried cortex, or bark, of a plant from the Magnoliaceae family, such as Magnolia officinalis, or a synthetic or semi-synthetic equivalent of such an extract or an active component or compound thereof. Typically, extracts of Magnolia Cortex (the bark of Magnolia officinalis) contain hydrophobic compounds including magnolol, honokiol, tetrahydromagnolol, and tetrahydrohonokiol. According to some embodiments, the antibacterial plant extract is active against bacteria selected from, but not limited to, Actinobacillus actinomycetemcomitans, Porphyromonas gingivalis, Prevotella intermedia, Micrococcus luteus and Bacillus subtilis.

According to some embodiments, the magnolia extract is made from dried Magnolia plant bark and can be prepared by extracting the bark using an appropriate solvent. Exemplary solvents include compatible liquids, such as hydrocarbons and substituted hydrocarbons.

According to some embodiments, an effective concentration of magnolia bark extract is a concentration that results in a reduction of oral or periodontal bacteria in a subject treated with an oral care composition comprising magnolia bark extract, when compared with the composition without the magnolia bark extract component.

According to some embodiments, the extract active ingredients used in the oral care compositions of the described invention are reproducible, stable, and have microbiological safety. According to some embodiments, the magnolia extract is isolated by supercritical fluid extraction (SFE) using carbon dioxide (CO₂). Supercritical fluids use a solvent that is readily available, inexpensive, and environmentally safe (such as CO₂). Carbon dioxide is non-toxic, non-explosive, readily available and easily removed from the extracted products.

According to some embodiments, SFE extraction produces a much lighter color of magnolia extract (a light beige product) for an aesthetically pleasing oral composition formulation.

According to some embodiments, the active ingredient in the magnolia extract comprises either magnolol, honokiol, or both. Magnolol and honokiol are non-ionic hydroxybiphenyl compounds. Additionally, tetrahydromagnolol and tetrahydrohonokiol are hydrogenated analogs of magnolol and honokiol is often found in relatively small concentrations in the extracts of magnolia, and as such may be included in the composition.

Thus, according to some embodiments, the magnolia extract comprises one or more of the hydrophobic compounds: magnolol, honokiol, tertrahydromagnolol, and tetrahydrohonokiol, and includes mixtures thereof.

According to some embodiments, the magnolia extract of the described invention comprises magnolol, honokiol, or both in an amount of about 2% to about 99% by weight. According to some embodiments, the magnolia extract comprises magnolol, honokiol, or both in an amount greater than 50% by weight. According to some embodiments, the magnolol is present in an amount greater than 50% by weight (i.e., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%), greater than 70% by weight (i.e., at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%), or greater than 90% by weight (i.e., at least 90%, 91%, 92%, 93%, 94%, 95%, 96%, 97%, 98%, or 99%). In another embodiment, honokiol is present in an amount less than 50% by weight (less than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, or lower), in an amount less than 30% by weight (i.e., less than 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, or lower), in an amount less than 10% by weight (i.e., less than 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, or lower).

The MBE may be present in the oral composition in an amount of from about 0.001% to about 10% by weight. According to some embodiments, the MBE is present in the oral composition in an amount of about 0.001% to about 5.0% by weight (at least 0.001%, 0.002%, 0.003%, 0.004%, 0.005%, 0.006%, 0.007%, 0.008%, 0.009%, 0.10%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5%), or about 0.001% to about 2.0% by weight (at least 0.001%, 0.002%, 0.003%, 0.004%, 0.005%, 0.006%, 0.007%, 0.008%, 0.009%, 0.10%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0%). According to another embodiment, the MBE is present in the oral composition in an amount of about 1.0 to about 2.0% by weight (i.e., at least 1.0%, 1.1%, 1.2%, 1.3%, 1.4%, 1.5%, 1.6%, 1.7%, 1.8%, 1.9%, or 2.0%). According to other embodiments, the MBE is present in amounts less than 1% by weight, for example the MBE may be present in the oral composition in an amount of from about 0.01 to about 1% by weight (i.e., no more than 0.001%, 0.002%, 0.003%, 0.004%, 0.005%, 0.006%, 0.007%, 0.008%, 0.009%, 0.10%, 0.2%, 0.3%, 0.4%, 0.5%, 0.6%, 0.7%, 0.8%, 0.9%, or 1.0%). According to one embodiment, the MBE is present in an amount from about 0.01 to 0.5% by weight (e.g., no more than 0.10%, 0.2%, 0.3%, 0.4%, or 0.5%). According to some embodiments, the oral compositions will comprise from about 1 to about 20 mg of MBE (i.e., 1 mg, 1.5 mg, 2 mg, 2.5 mg, 3 mg, 3.5 mg, 4 mg, 4.5 mg, 5.0 mg, 5.5 mg, 6 mg, 6.5 mg, 7.0 mg, 7.5 mg, 8 mg, 8.5 mg, 9 mg, 9.5 mg, 10 mg, 10.5 mg, 11.0 mg, 11.5 mg, 12,0 mg, 12.5 mg, 13.0 mg, 13.5 mg, 14 mg, 14.5 mg, 15.0 mg, 15.5 mg, 16.0 mg, 16.5 mg, 17.0 mg, 17.5 mg, 18.0 mg, 18.5 mg, 10.0 mg, 19.5 mg, or 20 mg). In another aspect, the oral compositions will comprise from about 1 to about 10 mg of MBE. (i.e., 1 mg, 1.5 mg, 2 mg, 2.5 mg, 3 mg, 3.5 mg, 4 mg, 4.5 mg, 5.0 mg, 5.5 mg, 6 mg, 6.5 mg, 7.0 mg, 7.5 mg, 8 mg, 8.5 mg, 9 mg, 9.5 mg, or 10 mg).

### Xylitol

Xylitol is well known to inhibit the growth of Streptocococcus mutans, an oral bacteria implicated in plaque formation, and it is known that this inhibition causes reduced acid formation, which in turn is believed to inhibit caries formation. Without being limited by theory, Xylitol serves not only as a sweetener, but also as an additive that helps inhibit the growth of bacteria in the mouth. In chewing gum, for example, Xylitol supports increased mineralization and that helps strengthen enamel. Since thin enamel is one cause of sensitive teeth, including xylitol in an oral care routine fortifies this layer, decreasing the risk of nerve exposure, and painful sensitivity, on the teeth's pulp. Xylitol also increases salivary flow. The minerals that are present in saliva then work to remineralize your enamel. Another benefit of xylitol is that while it shares the sweetening effect of sugar, xylitol is not an energy source for a particular bacteria that is found in oral biofilm, according to a study published in the Archives of Oral Biology. Thus, xylitol can starve the harmful bacteria in the mouth, reducing plaque buildup and tooth decay. This can help prevent dental caries and inflammatory gum diseases.

According to some embodiments, xylitol is present in the oral care composition in an amount of from about 0.5% to about 95%, by total weight of the oral care composition, for example at least 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%, by total weight of the oral care composition.

### Carboxylic Block Polymers

According to some embodiments, the oral care composition comprises a carboxylic block polymer.

The GANTREZ series of copolymers of monoalkyl esters of poly (methyl vinyl ether/maleic acid) with varying ester groups form tough, clear glossy films that exhibit tack-free adhesion, have excellent substantivity, and moisture resistance. Film properties and solubility can be modified by the type and degree of neutralization, allowing formulation across product lines from regular hold to super hold and from a natural, soft feel to a firmer, harder holding finish. Supplied as clear, viscous solutions, they are soluble in alcohols, esters, ketones and glycol ethers and have good compatibility with aerosol propellants.

According to one embodiment, the carboxylic block polymer is a water-soluble methyl vinyl ether and maleic acid copolymer. Such block polymers are effective in delivery and retention of active ingredients including antimicrobials, flavors, coolants, and medicants in toothpaste and mouthwash applications. According to some embodiments, the methyl vinyl ether and maleic acid copolymer is a commercially available Gantrez®S polymer (Ashland Specialty Chemical). For example, GANTREZ S-97 BF helps control the formation and growth of tartar. GANTREZ S-97 BF is commercially available in a white powder form.

According to one embodiment, a carboxylic block polymer is present in the oral care composition in an amount of from about 0.5% to about 95%, by total weight of the oral care composition, for example 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, by total weight of the oral care composition.

### Acrylic Acid Metacrylic Acid Copolymers

According to some embodiments, the polymer comprises an anionic polymer or copolymer. According to some embodiments, the anionic polymer comprises acrylic acid, methacrylic acid, or a copolymer of acrylic acid and metacrylic acid. According to some embodiments, the anionic polymer comprises amino alkyl methacrylate copolymers, methacrylic ester copolymers, metacrylic acid copolymers, or ammonioalkyl methacrylate copolymers. A large number of esters of methacrylic acid have been reported in the literature. Classically, methyacrylic esters of primary and secondary alcohols can be prepared by direct esterification with glacia 1methacrylic acid; esters of tertiary alcohols or phenols can be prepared by reacting them with methacrylyl chloride. According to some embodiments, the anionic polymer is commercially available from Evonik Industries as EUDRAGIT®. For example, EUDRAGIT® L, S, FS and E polymers with acidic or alkaline groups enable pH-dependent release.

According to one embodiment, an anionic polymer or copolymer is present in the oral care composition in an amount of from about 0.5% to about 95%, by total weight of the oral care composition, for example 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, by total weight of the oral care composition.

### Sweeteners

The oral care compositions of the described invention can also comprise a sweetener. The sweeteners can be natural or artificial. Non-limiting examples of sweeteners can include nutritive sweeteners, sugar alcohols, synthetic sweeteners, high intensity natural sweeteners, and combinations thereof. According to one example, oral care compositions can comprise a nutritive sweetener and a synthetic sweetener. According to one example, the oral care compositions can comprise from about 0.05% to about 80% sweetener, from about 0.05% to about 70% sweetener, from about 0.1% to about 60% sweetener, from about 1% to about 50% sweetener, and in another example from about 5% to about 45% sweetener.

Non-limiting examples of nutritive sweeteners can include sucrose, dextrose, glucose, fructose, lactose, tagatose, maltose, trehalose, and combinations thereof. According to one example, the oral care compositions can comprise sucrose. According to one example the oral care compositions can comprise from about 10% to about 80% nutritive sweetener, according to another example from about 10% to about 70% sweetener, according to another example from about 20% to about 60% nutritive sweetener, according to another example about 30% to about 55% nutritive sweetener, according to another example about 35% to about 50% nutritive sweetener, according to another example about 40% to about 48% nutritive sweetener, and according to another example about 42% to about 47% nutritive sweetener.

According to one example, the oral care compositions may not comprise a nutritive sweetener. According to one example, the sweetener can comprises a sweetener selected from the group comprising sugar alcohols, synthetic sweeteners, high intensity natural sweeteners, and combinations thereof. The dosage forms can be sold as reduced sugar, lower in sugar, low in sugar, or sugar free.

Non-limiting examples of sugar alcohols can include xylitol, sorbitol, mannitol, maltitol, lactitol, isomalt, erythritol, and combinations thereof. According to one example the dosage form can comprise from about 10% to about 80% sugar alcohol, from about 10% to about 70% sugar alcohol, according to another example from about 20% to about 60%, according to another example about 30% to about 55%, according to another example about 35% to about 50%, according to another example about 40% to about 48%, and according to another example about 42% to about 47%.

Non-limiting examples of synthetic sweeteners can include aspartame, acesulfame potassium, alitame, sodium saccharin, sucralose, neotame, cyclamate, and combinations thereof. According to one example, the dosage forms comprise sucralose. According to another example the dosage forms can comprise both sucralose and sucrose. According to one example the dosage form can comprise from about 0.01% to about 10% artificial sweetener, according to another example from about 0.05% to about 5%, according to another example from about 0.08% to about 3%, and according to another example from about 0.09% to about 1%, according to another example from about 0.1% to about 0.5%, and according to another example about 0.2% to about 0.25%.

Non-limiting examples of high intensity natural sweeteners can include neohesperidin dihydrochalcone, stevioside, rebaudioside A, rebaudioside C, dulcoside, monoammonium glycrrhizinate, thaumatin, and combinations thereof. According to one example the dosage form can comprise from about 0.01% to about 10% high intensity natural sweeteners, according to another example from about 0.05% to about 5%, according to another example from about 0.08% to about 3%, according to another example from about 0.09% to about 1%, according to another example from about 0.1% to about 0.5%, and according to another example about 0.2% to about 0.25%.

### Effervescent Agents

According to certain embodiments, the oral care composition further comprises an effervescent agent. When the consumer places the dosage form, for example a chewable tablet, into the oral cavity, the dosage form is effective to effervesce, creating a sensory feel of a clean mouth. According to certain embodiments, the effervescent ingredient comprises a carbonate or bicarbonate, including but not limited to, sodium bicarbonate, sodium carbonate or ammonium bicarbonate and the like. It is contemplated by the present disclosure that the effervescent agent dissolves in the saliva in the mouth to react and generate carbon dioxide in a “burst” in the mouth. The effervescence can provide a consistent, gentle, steady release of small bubbles, similar to champagne. The effervescence can also provide a signal to the consumer that the product is working.

### Abrasives

The oral care compositions of the described invention may further comprise an abrasive or abrasive polishing material. In certain embodiments, the abrasive is a mineral abrasive.

The abrasive or abrasive polishing material can be any material that does not excessively abrade dentin. The oral care compositions of the described invention may comprise abrasive polishing material in an amount of from about 6% to about 70% or from about 10% to about 50%, by weight of the oral care composition. The composition can contain from about 2% to about 25% abrasive polishing material, alternatively from about 5% to about 20%, alternatively from about 7% to about 18%, alternatively from about 9% to about 16%, and alternatively from about 12% to about 15%. The composition can contain 10% abrasive polishing material and alternatively about 15% abrasive polishing material.

Typical abrasive polishing materials can include silicas including gels and precipitates; aluminas; phosphates including orthophosphates, polymetaphosphates, and pyrophosphates; and mixtures thereof. Specific examples include silicone microspheres such as polyorganosilsesquioxane particles, dicalcium orthophosphate dihydrate, calcium pyrophosphate, tricalcium phosphate, calcium polymetaphosphate, insoluble sodium polymetaphosphate, rice hull silica, hydrated alumina, beta calcium pyrophosphate, calcium carbonate, and resinous abrasive materials such as particulate condensation products of urea and formaldehyde, and others such as disclosed by Cooley et al in U.S. Pat. No. 3,070,510, incorporated by reference in its entirety herein.

According to some embodiments, the oral care composition can contain a silica abrasive. Silica abrasive polishing materials that may be used in the present disclosure, as well as other abrasives, generally have an average particle size ranging between about 0.1 to about 30 μm or from about 5 to about 15 μm. The abrasive can be precipitated silica or silica gels such as the silica xerogels described in Pader et al., U.S. Pat. No. 3,538,230 and DiGiulio, U.S. Pat. No. 3,862,307. Silica xerogels marketed under the trade name “Syloid” by the W.R. Grace & Company, Davison Chemical Division, Augusta, Ga. may be used. Also precipitated silica materials such as those marketed by the J. M. Huber Corporation, Edison, N.J. under the trade name, “Zeodent”, for example the silica carrying the designation “Zeodent 119”, may be used. The types of silica dental abrasives useful in the oral care compositions of the described invention are described in more detail in U.S. Pat. Nos. 4,340,583; 5,589,160; 5,603,920; 5,651,958; 5,658,553; and 5,716,601.

The abrasive may include polymethyl organosiloxane particles. The types of polymethyl organosiloxane particles useful in the oral care compositions of the described invention are described in more detail in U.S. Pat. No. 9,017,647. It may be advantageous to select an abrasive containing polymethyl organosiloxane particles, because they are less reactive with ingredients commonly found in oral care compositions, in including oral care actives.

The abrasive may include calcium pyrophosphate. The abrasive may include poly(methyl methacrylate), calcium carbonate, dicalcium phosphate, and/or barium sulfate.

According to some embodiments, the abrasive is selected from the group consisting of silicic acids, calcium carbonates, calcium phosphates, aluminum oxides and/or hydroxyapatites, sodium metaphosphate, potassium metaphosphate, tricalcium phosphate, dihydrated dicalcium phosphate, micronized silicon, aluminum silicate, calcined alumina, bentonite, surface-active substances (e.g., sodium lauryl sulfate, sodium lauryl sarcosinate, and cocamidopropylbetaine), and other siliceous materials, and combinations thereof.

### Flavorants

Taste is an important attribute, especially in a chewable tablet. According to some embodiments, an oral care composition of the described invention may include a flavorant. Examples of some traditional flavorants or flavor compounds that may be used in the oral care compositions are mint oils, and components thereof, wintergreen, clove bud oil, cassia, sage, parsley oil, marjoram, lemon, orange, propenyl guaethol, heliotropine, cis-4-heptenal, diacetyl, methyl-.rho.-tert-butyl phenyl acetate, methyl salicylate, ethyl salicylate, 1-menthyl acetate, oxanone, alpha-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, .alpha.-terpineol, linalool, limonene, citral, neral, geranial, geraniol nerol, maltol, ethyl maltol, anethole, dihydroanethole, carvone, menthone, beta-damascenone, ionone, gamma.-decalactone, gamma-nonalactone, .gamma.-undecalactone, isopulegol, piperitone, or combinations thereof. Generally suitable flavoring ingredients are chemicals with structural features and functional groups that are less prone to redox reactions. These include derivatives of flavor chemicals that are saturated or contain stable aromatic rings or ester groups. Flavor components can be present in an amount of from about 0.4% to about 5%, by total weight of the oral care composition, according to another example from about 0.8% to about 4%, according to another example from about 1% to about 3.5%, and According to another example from about 1.5% to about 3%. It can be desirable to have a flavor composition at less than about 4%, less than about 3.5%, by total weight of the oral care composition, according to another example less than about 3%, and according to another example less than about 2%.

### Sensates

According to some embodiments, an oral care composition of the described invention may include a sensate. Sensate agents or molecules are intended to deliver a cooling, warming, or tingling sensation. The most well-known cooling sensate compound can be menthol, particularly L-menthol, which is found naturally in peppermint and spearmint oils notably of Mentha piperita, Mentha arvensis L and Mentha viridis L. L-menthol has a characteristic peppermint odor, has a clean fresh taste and exerts a cooling sensation when applied to the skin and mucosal surfaces. Some examples of warming sensates include ethanol; capsicum; nicotinate esters, such as benzyl nicotinate; polyhydric alcohols; capsicum powder; a capsicum tincture; capsicum extract; capsaicin; homocapsaicin; homodihydrocapsaicin; nonanoyl vanillyl amide; nonanoic acid vanillyl ether; vanillyl alcohol alkyl ether derivatives such as vanillyl ethyl ether, vanillyl butyl ether, vanillyl pentyl ether, and vanillyl hexyl ether; isovanillyl alcohol alkyl ethers; ethyl vanillyl alcohol alkyl ethers; veratryl alcohol derivatives; substituted benzyl alcohol derivatives; substituted benzyl alcohol alkyl ethers; vanillin propylene glycol acetal; ethyl vanillin propylene glycol acetal; ginger extract; ginger oil; gingerol; zingerone; or combinations thereof. Warming sensates may be included in an oral care composition for example at a level of about 0.01% to about 5%, or for example at a level of about 0.01% to about 4%, or for example at a level of about 0.01% to about 2% by weight of the oral care composition.

### Colorants

According to some embodiments, an oral care composition of the described invention may include a colorant. Examples of some colorants that may be used in oral care compositions include D&C Yellow No. 10, FD&C Blue No. 1, FD&C Red No. 40, D&C Red No. 33 and combinations thereof. According to certain examples, the composition comprises a colorant in an amount of from about 0.0001% to about 0.1% or from about 0.001% to about 0.01%, by weight of the oral care composition. Some colorants provide an unwanted taste, for example, D&C Red No. 33. The unwanted tastes often associated with this colorant are metallic, sharp, or chemical. Colorants are generally present in an amount of from about 0.001% to about 0.5%, by weight of the oral care composition.

### Desensitizing Agents

According to some embodiments, an oral care composition of the described invention may include a desensitizing agent. For example nitrate salt, a bicarbonate salt, potassium nitrate, arginine-bicarbonate-phytate complex, potassium citrate or oxalate and arginine may be added to the oral care composition.

### Anti-Oxidants

According to some embodiments, an oral care composition of the described invention may include an anti-oxidant. According to some embodiments, the anti-oxidant can be chosen from: naturally occurring tocopherols and their derivatives (e.g., Vitamin E acetate), Vitamin C and its salts and derivatives (e.g., ascorbyl palmitate, Mg ascorbyl phosphate, ascorbyl acetate), Vitamin A and derivatives (Vitamin A palmitate), tocotrienols, flavonoids, alpha-hydroxy acids (e.g., citric acid, lactic acid, malic acid, tartaric acid) and their Na, Ka and Ca salts, flavonoids, quercetin, phenolic benzylamines, propyl gallate, octyl gallate, dodecyl gallate, butylhydroxyanisole (BHA, E320), butylhydroxytoluene (BHT, 2,6-di-tert.-butyl-4-methylphenol, E321), lecithins, mono- and diglycerides of edible fatty acids esterified with citric acid, carotenoids, carotenes (e.g., .alpha.-carotene, .beta.-carotene, lycopene) and their derivatives, phytic acid, lactoferrin, EDTA, EGTA), folic acid and its derivatives, ubiquinone and ubiquinol and their derivatives, ferulic acid and its derivatives, zinc and its derivatives (e.g., ZnO, ZnSO₄), selenium and its derivatives (e.g., selenium methionine), orthophosphates and Na, K and Ca salts of mono-phosphoric acids, and constituents, extracts and fractions thereof isolated from plants, (e.g., tea, green tea, algae, grapeseeds, wheat germ, chamomile, rosemary, oregano), and combinations thereof.

### Anti-Caries Agents

According to some embodiments, an oral care composition of the described invention may include an anti-caries agent. According to some embodiments, the anti-caries agent is a fluoride ion source selected from: inorganic fluoride salts, such as soluble alkali metal, alkaline earth metal salts (e.g., sodium fluoride, stannous fluoride, potassium fluoride, ammonium fluoride, calcium fluoride), a copper fluoride such as cuprous fluoride, zinc fluoride, barium fluoride, sodium fluorosilicate, ammonium fluorosilicate, sodium fluorozirconate, ammonium fluorozirconate, sodium monofluorophosphate, aluminum mono- and di-fluorophosphate, fluorinated sodium calcium pyrophosphate, and combinations thereof.

### Charcoal

According to some embodiments, an oral care composition of the described invention may include charcoal, for example activated charcoal. The charcoal may be a powder.

Any other mouth-feel agents may be included if desired, in the oral care compositions of the described invention.

The oral care compositions of the described invention may also include a saliva-stimulating agent.

While ingredients are sometimes identified herein by category, e.g. a flavorant, a pigment, a dye, a whitening agent, an anti-tartar agent, a desensitizing agent, a sensate, a vitamin, a preservative, an enzyme, saliva-stimulating agent, this identification is for convenience and clarity, but is not intended to be limiting. All of the ingredients in the compositions may have functions in addition to their primary function, and may contribute to the overall properties of the composition, including its stability, efficacy, consistency, mouthfeel, taste, odor and so forth.

In certain embodiments, the components of the oral care compositions of the disclosure are distributed evenly. In other embodiments, the components of the oral care compositions of the disclosure are distributed in layers throughout the tablet.

According to one embodiment, the dosage form is a chewable multi-layer tablet. In one example, one layer is effervescent and the other layer is not effervescent. In another example, one layer has a first component of the composition and another layer has a different component of the composition. In another example, the multi-layer tablet comprises one layer that has a first flavor and another layer that has a second flavor.

The average bonding time of the tablets of the described invention is between about 2 hours to about 15 hours, for example 5-10 hours, or about 12 hours. According to one embodiment, the average bonding time of the tablets of the described invention is for example 1, 2, 3, 4, 5, 6, 7, 8, 9, 10, 11, 12, 13, 14, or 15 hours. According to certain embodiments, the bonding time is about 12 hours.

According to some embodiments, the described invention relates to oral care compositions that are in tablet form, particularly in chewable tablet form. Thus, the chewable tablets of the disclosure can be orally administered without water. As the consumer chews, the dosage form can easily fracture and some portions of the dosage form can dissolve in the oral cavity. The dosage form of the disclosure is digestible.

The tablet can be any shape. Non-limiting examples of shapes can include round, oblong, oval, square, rectangular, diamond, triangular, five-sided, six-sided, seven-sided, eight-sided, irregular, or combinations thereof. The tablet of the described invention can be shiny or matte.

According to certain embodiments, the tablet comprises a contoured surface. The contoured surface provides multi-level touch points to provide a deeper clean around each tooth.

The dosage form can be any size. According to one example, the dosage form is a size that can easily fit inside the oral cavity of the intended subject. According to one example the dosage form has a surface area from about 300 mm² to about 1300 mm², according to another example from about 400 mm² to about 1000 mm², in another example from about 500 mm² to about 900 mm², according to another example from about 600 mm² to about 800 mm², according to another example from about 625 mm² to about 720 mm², and according to another example from about 650 mm² to about 700 mm². According to one example, the dosage form is circular or oval and the largest radius is from about 5 mm to about 30 mm, according to another example from about 8 mm to about 25 mm, according to another example about 10 mm to about 20 mm, and according to another example about 13 mm to about 18 mm. According to another example, the depth which is perpendicular from the radius, as measured from the highest point on the dosage form, is from about 2 mm to about 20 mm, according to another example from about 3 mm to about 15 mm, according to another example about 3.5 mm to about 10 mm, according to another example about 4 mm to about 7 mm, and according to another example about 4.5 mm to about 5.5 mm.

According to certain embodiments, the tablet may be between about 0.25 inches to 0.75 inches in diameter, for example 0.25, 0.26, 0.30, 0.35, 0.40, 0.45, 0.50, 0.55, 0.60, 0.65, 0.75 inches in diameter.

According to other embodiments, the tablet may be between about 0.1 inches to 0.5 inches thick, for example 0.1, 0.15, 0.2, 0.25, 0.3, 0.35, 0.4, 0.45, 0.5 inches thick.

According to certain embodiments, the tablet is about 0.5 inches in diameter and 0.25″ thick.

Optionally, the tablets of the described invention may comprise a coating, meaning a dry outer layer of material applied to the surface of the dosage form. Solid dosage forms are coated for a number of reasons, including for controlling the release profiles of the dosage form. Advantages of tablet coating include, without limitation, taste masking, odor masking, physical protection, and chemical protection. Techniques for tablet coating include sugar coating, film coating, and enteric coating. There also are coating methods that overcome the disadvantages associated with solvent based coatings in which coating materials are directly applied onto the surface of the solid dosage form without using any solvent. These include, for example, electrostatic dry coating, magnetically assisted impaction coating, compression coating, hot melt coating, powder coating, and supercritical fluid coating, all of which are within the scope of this disclosure.

According to one embodiment, the coating comprises an anionic polymer or copolymer. According to one embodiment, the coating comprises about 2% to about 10%, about 2% to about 8%, about 2% to about 5%. According to one embodiment, the coating comprises about 2%, 2.1%, 2.2%, 2.3%, 2.4%, 2.5%, 2.6%, 2.7%, 2.8%, 2.9%, 3.0%, 3.1%, 3.2%, 3.3%, 3.4%, 32.5%, 3.6%, 3.7%, 3.8%, 3.9%, 4%, 4.1%, 4.2%, 4.3%, 4.4%, 4.5%, 4.6%, 4.7%, 4.8%, 4.9% or 5% anionic polymer or copolymer by total weight. According to another embodiment, the anionic polymer comprises acrylic acid, or methacrylic acid. According to another embodiment, the anionic polymer is a copolymer of acrylic acid and methacrylic acid, a copolymer of esters of acrylic acid, a copolymer of esters of methacrylic acid, or a copolymer of esters of acrylic acid and esters of methacrylic acid.

Exemplary coating compositions are described in U.S. Pat. No. 9,668,977, incorporated by reference in its entirety herein.

The tablets of the described invention have a certain hardness and friability (meaning the tendency to chip, crumble or break following compression. The tablet dosage forms can be hard enough to withstand the rigors of handling and transportation experienced in the manufacturing plant, in the drug distribution system, and in the field in the hands of the consumer. If the dosage form is too soft it can fall apart or get crushed into a powder or pieces before the consumer can consume it. Conversely, if the dosage form is too hard it can be difficult to chew and it can produce more sound when it is chewed. Furthermore, hardness is also an important characteristic to provide the correct mouthfeel for a chewable product i.e., if the product is too soft or too hard it may not have the correct texture when chewed.

Tablet breaking force is a measure of hardness and can be measured using USP Test Method 1217 using a Vankel Benchsaver VK200 Tablet Hardness Tester. Friability can be measured using USP Test Method 1216. The dosage forms can have lower friability than antacids, which means that the solid dosage form can be reduced to smaller pieces with less effort, which can ultimately lead to a faster dissolution of the dosage form.

### III. Methods of Use

The oral care compositions of the described invention can be used in a number of methods.

According to another aspect, the described invention provides a method for cleaning surfaces of the oral cavity comprises contacting the oral cavity with the oral care composition of any of the aspects and embodiments described herein. According to one embodiment, the oral cavity includes the teeth, the tongue, the gums and the cheeks.

According to another aspect, the described invention provides a method for the treatment or prevention of enamel erosion on a dental surface, comprising contacting the dental surface with the oral care composition of any of the aspects and embodiments described herein.

Dental plaque is a biofilm that adheres to tooth and other oral surfaces, particularly at the gingival margin, and is implicated in the occurrence of gingivitis, periodontitis, caries and other forms of periodontal disease. Dental plaque is cohesive and highly resistant to removal from teeth and/or oral surfaces. Dental plaque comprises glucans, which are insoluble polysaccharides that provide plaque with its cohesive properties. The bacterial enzyme glucosyltransferase converts dietary sugar into glucans. Plaque mineralizes to form a hard deposit called calculus (or tartar), which becomes a local irritant for the gums, causing gingivitis.

Everyday activities such as smoking or other oral use of tobacco products, and eating, chewing or drinking certain foods and beverages (in particular coffee, tea, coke, and red wine), cause undesirable staining of surfaces of teeth. Staining can also result from microbial activity, including that associated with dental plaque. The chromogens or color-causing substances in these materials become part of the pellicle layer and can permeate the enamel layer of the tooth. Even with regular brushing and flossing, years of chromogen accumulation can impart noticeable tooth discoloration. Thus, according to another aspect, the described invention provides a method for the treatment or inhibition of a chemical stain, plaque, and/or tartar on a dental surface, comprising contacting the dental surface with the oral care composition of any of the aspects and embodiments described herein. According to another aspect, the described invention provides a method for whitening the teeth, comprising contacting the dental surface with the oral care composition of any of the aspects and embodiments described herein.

According to another aspect, the described invention provides a method for the treatment or inhibition of gum disease comprising contacting the oral cavity with the oral care composition of any of the aspects and embodiments described herein. According to one embodiment, the gum disease is gingivitis. According to another embodiment, the gum disease is periodontitis.

One of the major contributors to malodor in the oral cavity is the bacteria present on the soft and hard, oral tissues. Manual brushing and rinsing help to remove the bacteria, but they eventually repopulate over a period of time. Thus, according to another aspect, the described invention provides a method for the treatment or inhibition of halitosis comprising contacting the oral cavity with the oral care composition of any of the aspects and embodiments described herein.

Biofilms form when bacteria adhere to surfaces in some form of watery environment and begin to excrete a slimy, glue-like substance that can stick to all kinds of materials—metals, plastics, soil particles, medical implant materials, biological tissues. Biofilms can be formed by a single bacterial species, but biofilms more often consist of many species of bacteria, as well as fungi, algae, protozoa, debris, and corrosion products. Essentially, a biofilm may form on any surface exposed to bacteria and some amount of water. Dental plaque is a yellowish biofilm that builds up on the teeth. Biofilms contain communities of disease-causing bacteria and their uncontrolled accumulation has been associated with cavities and gum disease (both gingivitis and periodontitis). Accordingly, according to another aspect, the described invention provides a method for inhibition of biofilm formation on a dental surface comprising contacting the oral cavity with the oral care composition of any of the aspects and embodiments described herein.

According to another aspect, the described invention provides a method for the treatment or inhibition of bacteria from sticking together and growing into bigger colonies in an oral cavity comprising contacting the oral cavity with the oral care composition of any of the aspects and embodiments described herein.

The oral care composition of the described invention can be used one time per day or multiple times per day. The oral care composition can be used on a daily basis or only as needed. According to one example, the oral care composition can be used after a meal, snack, or beverage. According to another example, the oral care composition can be used about 30 minutes, about 60 minutes, about 90 minutes, or about 120 minutes after eating. According to another example, the oral care composition can be used without food. According to another example, the oral care composition can be used without water.

According to one example a consumer can ingest (meaning take into the body orally) one chewable tablet per dose, according to another example two chewable tablets per dose, in another example three chewable tablets per dose, and according to another example four chewable tablets per dose. According to one example, the consumer can ingest at least one dose per day, according to another example at least two doses per day, according to another example at least three doses per day, and according to another example at least four doses per day. According to one example, the doses can be taken one to twelve times per day, according to another example two to ten times per day, according to another example four to six times a day, and according to another example three to four times per day. According to one example the doses can be taken on an as-needed basis. According to some embodiments, the doses can be packaged in a sterile unit dose package, meaning a sterile individual packet containing one tablet per package. According to some embodiments, the sterile unit dose package can be presented in a strip containing more than two unit dose package. According to some embodiments, the strip is perforated so as to enable simple separation of each unit dose package from the other unit dose packages in the strip. According to some embodiments, the sterile unit dose package can be presented in a roll of sterile unit dose packages. According to some embodiments, the roll is perforated so as to enable simple separation of each unit dose package from the other unit dose packages in the roll. According to some embodiments, the sterile unit dose package is recyclable. According to some embodiments, the recyclable sterile unit dose packaging is a recyclable sachet.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any disclosure disclosed or claimed herein or that it alone, or in any combination with any other reference or references, teaches, suggests or discloses any such disclosure. Further, to the extent that any meaning or definition of a term in this document conflicts with any meaning or definition of the same term in a document incorporated by reference, the meaning or definition assigned to that term in this document shall govern.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, exemplary methods and materials have been described.

### Examples

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Described herein is an exemplary oral care composition (e.g. a delivery system, preparation, or product for oral hygiene and oral health).

The characteristics of the oral care composition include the following:
Adherent to surfaces of the oral cavity, including teeth, tongue, cheeks, and gums
Chewable
Pleasant taste
Mildly abrasive
Digestible
Water-free
Toothbrush-free
Polishing agent-free
Activated in situ by chewing action and by contacting saliva
Contoured surface
Portable

According to one embodiment, the composition contains:
Neem or coconut oil
Xylitol
Carboxylic block copolymers
A mineral abrasive, e.g., calcium carbonate or micronized silicon
Optionally, charcoal
Optionally magnolia bark extract

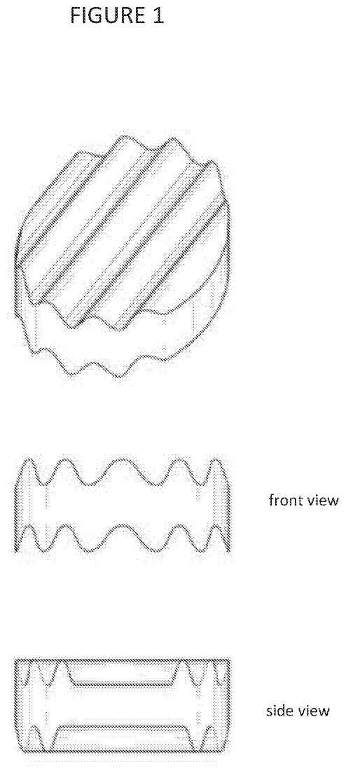

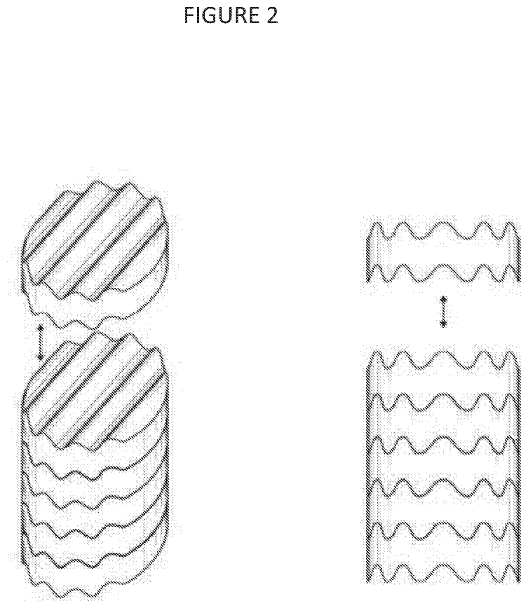

According to one embodiment, the size of the oral care composition is about 0.5″ diameter, 0.25″ thick. The ingredients may be distributed evenly or in layers, as shown in FIGS. 1 and 2. The tablet may have a coating.

During chewing and contact with saliva, the ingredients are immediately released. The composition bonds to surfaces and crevices of the oral cavity for up to 12 hours and a minimum of 3 hours.

According to one embodiment, the benefits of the oral care compositions are as follows:
Effective for cleaning surfaces of the oral cavity including teeth, tongue, gums, cheeks
Effective for prevention of tooth erosion, where block polymers bind to surface enamel and physically block acid attack
Reducing tooth staining
Refreshes breath
Antibacterial
Nonirritating
Reduces or prevents inflammation

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A water-free, and rinse-free oral care composition in form of a tablet, comprising:
0. 5%-95% neem;
0. 5%-95% xylitol;
0. 5%-95% one or more polymers, and
0.01%-5% sensate,
wherein the tablet is activated in situ by a chewing action and/or by contacting saliva and the sensate component provides a sensorial feel of a clean mouth.

2. The oral care composition of claim 1, further comprising an effervescent agent.

3. The oral care composition of claim 1, wherein the composition is digestible.

4. The oral care composition of claim 1, wherein the polymer is a carboxylic block polymer.

5. The oral care composition of claim 4, wherein the carboxylic block polymer is GantrezS-97BF.

6. The oral care composition of claim 1, wherein the polymer is an anionic polymer.

7. The oral care composition of claim 6, wherein the anionic polymer comprises one or more of acrylic acid, an ester of acrylic acid, methacrylic acid or an ester of methacrylic acid.

8. The oral care composition of claim 7, wherein the anionic polymer is a copolymer of acrylic acid and methacrylic acid or a copolymer of an ester of acrylic acid and methacrylic acid.

9. The oral care composition of claim 1, further comprising an abrasive.

10. The oral care composition of claim 9, wherein the abrasive is a mineral abrasive.

11. The oral care composition of claim 9, wherein the abrasive is selected from the group consisting of: silicic acids, calcium carbonates, calcium phosphates, aluminum oxides and/or hydroxyapatites, sodium metaphosphate, potassium metaphosphate, tricalcium phosphate, dihydrated dicalcium phosphate, micronized silicon, aluminum silicate, calcined alumina, bentonite, surface-active substances (e.g., sodium lauryl sulfate, sodium lauryl sarcosinate, and cocamidopropylbetaine), and other siliceous materials, and combinations thereof.

12. The oral care composition of claim 1, further comprising charcoal.

13. The oral care composition of claim 1, further comprising 2-99% of an antibacterial plant extract.

14. The oral care composition of claim 13, wherein the antibacterial plant extract is magnolia bark extract (MBE).

15. The oral care composition of claim 1, wherein the tablet comprises a contoured surface.

16. The oral care composition of claim 1, wherein the tablet is between about 0.25 inches to 0.75 inches in diameter.

17. The oral care composition of claim 1, wherein the tablet is between about 0.1 inches to 0.5 inches thick.

18. The oral care composition of claim 1, wherein the tablet is about 0.5 inches in diameter and 0.25″ thick.

19. The oral care composition of claim 1, wherein the tablet comprises a coating.

20. The oral care composition of claim 1, wherein the neem component, xylitol component and one or more carboxylic block copolymers components are distributed evenly throughout the tablet.

21. The oral care composition of claim 1, wherein the neem component, xylitol component and one or more carboxylic block copolymers component are distributed in layers throughout the tablet.

22. The oral care composition of claim 1, wherein the composition further comprises one or more of a flavorant, a pigment, a dye, a whitening agent, an anti-tartar agent, a desensitizing agent, a sensate, a vitamin, a preservative, an enzyme, saliva-stimulating agent, or a mixture thereof.

23. A method for cleaning the surfaces of the oral cavity comprising contacting the oral cavity with the oral care composition of claim 1.

24. The method of claim 23, wherein the oral cavity includes the teeth, the tongue, the gums and the cheeks.

25. A method for the treatment or prevention of enamel erosion on a dental surface, comprising contacting the dental surface with the oral care composition of claim 1.

26. A method for the treatment or inhibition of a chemical stain, plaque, and/or tartar on a dental surface, comprising contacting the dental surface with the oral care composition of claim 1.

27. A method for the treatment or inhibition of gum disease comprising contacting the oral cavity with the oral care composition of claim 1.

28. A method for the treatment or inhibition of halitosis comprising contacting the oral cavity with the oral care composition of claim 1.

29. A method for the inhibition of biofilm formation on a dental surface comprising contacting the oral cavity with the oral care composition of claim 1.

30. A method for the treatment or inhibition of bacteria from sticking together and growing into bigger colonies in an oral cavity comprising contacting the oral cavity with the oral care composition of claim 1.
